(19) United States
(12) Patent Application Publication
(10) Pub. No.: US 2016/0152942 A1
(43) Pub. Date: Jun. 2, 2016

(54) METHOD FOR ISOLATING HIGHLY FURFURAL RESISTANT STRAIN AND STRAIN ISOLATED BY THE METHOD

(71) Applicant: KOREA INSTITUTE OF SCIENCE AND TECHNOLOGY, Seoul (KR)

(72) Inventors: Han Min WOO, Seoul (KR); Youngsoon UM, Seoul (KR); Gyeong Taek GONG, Seoul (KR); Dong Jin SUH, Seoul (KR); Yunje KIM, Seoul (KR)

(21) Appl. No.: 14/679,622

(22) Filed: Apr. 6, 2015

(30) Foreign Application Priority Data
Dec. 1, 2014 (KR) 10-2014-0169425

(51) Int. Cl. C12N 1/20

(52) U.S. Cl. CPC C12N 1/20

(57) ABSTRACT

A method for isolating a highly furfural-resistant strain is disclosed. The method includes (A) mixing soil with an isotonic solution and collecting the soil supernatant from the mixture, (B) diluting the soil supernatant and spreading the dilution on a medium supplemented with carboxymethyl cellulose as a nutrient source, and (C) growing a desired strain by culture in the medium spread with the diluted soil supernatant and isolating the strain. Also disclosed is a strain isolated by the method. The strain is Enterobacter cloacae strain GGT036 [Accession No. KCTC 12672BP].

### CROSS-REFERENCE TO RELATED APPLICATIONS

This application claims priority under 35 U.S.C. §119 to Korean Patent Application No. 10-2014-0169425 filed on Dec. 1, 2014 in the Korean Intellectual Property Office, the disclosure of which is incorporated herein by reference in its entirety.

### BACKGROUND OF THE INVENTION

1. Field of the Invention The present invention relates to a method for isolating Enterobacter cloacae strain GGT036 highly resistant to furfural, a toxic substance in hydrolysates, and a strain isolated by the method.

2. Description of the Related Art

Alternative energy sources to oil have been gaining interest due to global warming caused by increased carbon dioxide in the atmosphere and depletion of oil resources. Carbon neutral bioenergy has received particular attention as a source that has the potential to replace the petrochemical industry.

Bioenergy production based on the use of corn or sugar cane as first generation biomass has a serious disadvantage in that the food resource is diverted to energy production. In view of this disadvantage, the use of second generation non-edible lignocellulosic biomass resources is being actively investigated.

Lignocellulosic biomass is mainly composed of cellulose, hemicellulose, and lignin and can be converted into fuels, plastics, and other chemicals through microbial hydrolysis and fermentation.

Lignocellulosic biomass undergoes pretreatment at high temperature or pressure or using an acid to produce sugars, including glucose and xylose, which are readily available to microorganisms. At this time, however, various kinds of by-products are inevitably produced during hydrolysis as a pretreatment process.

The hydrolysis by-products include furfural, hydroxymethylfurfural (HMF), and acetic acid, which are responsible for low productivity in the fermentation of hydrolysates due to their toxicity to microorganisms.

Furfural derived from xylose is an important substance in hydrolysates that inhibits the microbial fermentation. The toxicity of hydrolysates is highly correlated with the concentration of furfural in the hydrolysates. Furfural is known to be converted to less toxic furfuryl alcohol in cells. This conversion requires the use of NADH or NADPH, and as a result, an imbalance in intracellular NAD(P)H level occurs, which is known to inhibit the growth of cells and the fermentation of hydrolysates.

Aside from this, furfural is known to cause genetic variation or weakens cell membranes and to interact with other substances (e.g., hydroxymethylfurfural and acetic acid) in hydrolysates, making its toxicity more severe.

Thus, there is a need for strains resistant to toxic substances, particularly furfural, in hydrolysates and methods for isolating the strains.

### PRIOR ART DOCUMENTS

### Patent Documents

Korean Patent No. 1165733

U.S. Patent Publication No. 2008/0090283

### Non-Patent Documents

Applied and environmental microbiology, 77 (15), 5132-5140

Journal of bioscience and bioengineering, 113 (4), 451-455

### SUMMARY OF THE INVENTION

It is an object of the present invention to provide a method for isolating Enterobacter cloacae strain GGT036 highly resistant to furfural, a toxic substance in hydrolysates.

It is a further object of the present invention to provide highly furfural-resistant Enterobacter cloacae strain GGT036 isolated by the method.

It is another object of the present invention to provide a method for producing bioenergy using Enterobacter cloacae strain GGT036 [Accession No. KCTC 12672BP].

According to one aspect of the present invention, there is provided a method for isolating a highly furfural-resistant strain, including (A) mixing soil with an isotonic solution and collecting the soil supernatant from the mixture, (B) diluting the soil supernatant and spreading the dilution on a medium supplemented with carboxymethyl cellulose as a nutrient source, and (C) growing a desired strain by culture in the medium spread with the diluted soil supernatant and isolating the strain.

The highly furfural-resistant strain is Enterobacter cloacae strain GGT036 [Accession No. KCTC 12672BP].

The medium may be supplemented with 3 to 10% by weight of carboxymethyl cellulose as a nutrient source.

According to a further aspect of the present invention, there is provided furfural-resistant Enterobacter cloacae strain GGT036 [Accession No. KCTC 12672BP].

Furfural has an IC₅₀ value of 45 to 55 mM against the Enterobacter cloacae strain GGT036 [Accession No. KCTC 12672BP].

The Enterobacter cloacae strain GGT036 [Accession No. KCTC 12672BP] may grow in a medium supplemented with carboxymethyl cellulose as a nutrient source.

According to another aspect of the present invention, there is provided a method for producing bioenergy, including inoculating Enterobacter cloacae strain GGT036 [Accession No. KCTC 12672BP] into a fermentation medium supplemented with a lignocellulosic hydrolysate as a carbon source and fermenting the lignocellulosic hydrolysate.

The lignocellulosic hydrolysate may be prepared by physical or chemical pretreatment of lignocellulosic biomass.

The Enterobacter cloacae strain GGT036 [Accession No. KCTC 12672BP] of the present invention is highly resistant to furfural, a toxic substance in lignocellulosic hydrolysates, and converts at least 80% of furfural to less toxic furfuryl alcohol.

In addition, the highly furfural-resistant strain of the present invention prevents growth inhibition and productivity reduction caused by toxic substances in lignocellulosic hydrolysates to produce metabolites, such as lactic acid, succinic acid, and sodium L-glutamate (MSG), as well as chemicals, such as biofuels and plastics, through fermentation of the hydrolysates. Therefore, the furfural-resistant strain of the present invention is helpful in efficient microbial fermentation.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is an optical microscopy image of Enterobacter cloacae strain GGT036 isolated in Example 1;

FIG. 2A is an image showing the growth of Enterobacter cloacae strain GGT036, E. coli, and Corynebacterium glutamicum in furfural-free media and FIG. 2B is an image showing the growth of Enterobacter cloacae strain GGT036, E. coli, and Corynebacterium glutamicum in furfural-containing media;

FIG. 3 is a graph showing the degrees of inhibition of growth of Enterobacter cloacae strain GGT036 in the presence of furfural at different concentrations; and

FIG. 4A is a graph showing changes in the concentration of furfural and furfuryl alcohol in a culture medium containing 20 mM furfural when Enterobacter cloacae strain GGT036 was allowed to grow in the culture medium, FIG. 4B is a graph showing changes in the concentration of furfural and furfuryl alcohol in a culture medium containing 40 mM furfural when Enterobacter cloacae strain GGT036 was allowed to grow in the culture medium, and FIG. 4C is a graph showing changes in the concentration of furfural and furfuryl alcohol in a culture medium containing 60 mM furfural when Enterobacter cloacae strain GGT036 was allowed to grow in the culture medium.

### DETAILED DESCRIPTION OF THE INVENTION

The present invention is directed to a method for isolating highly furfural-resistant Enterobacter cloacae strain GGT036 [Accession No. KCTC 12672BP] and a strain isolated by the method.

The present invention will now be described in detail. The present invention provides a method for isolating a highly furfural-resistant strain, including (A) mixing soil with an isotonic solution and collecting the soil supernatant from the mixture, (B) diluting the soil supernatant and spreading the dilution on a medium supplemented with carboxymethyl cellulose as a nutrient source, and (C) growing a desired strain by culture in the medium spread with the diluted soil supernatant and isolating the strain.

First, in step (A), a soil sample and a solution isotonic with the soil are mixed in a weight ratio of 1:80-150, the mixture is stirred, and the soil supernatant is collected.

The isotonic solution is a solution of 0.5 to 1.0 wt % NaCl, preferably 0.85 wt % NaCl, and allows a desired strain to maintain a steady state in the soil.

Next, in step (B), the collected soil supernatant is diluted 5- to 10-fold with a diluent and the dilution is then spread on a solid medium supplemented with carboxymethyl cellulose as a nutrient source.

The diluent is a solution of 0.5 to 1.0 wt % NaCl, preferably 0.85 wt % NaCl.

The dilution may be spread on a suitable medium, for example, a solid or liquid medium, to obtain colonies but is preferably spread on a solid medium to obtain a large amount of the highly furfural-resistant strain.

The solid medium is a medium containing carboxymethyl cellulose as a nutrient source. Specifically, the solid medium is prepared by mixing 100 parts by weight of a solid medium composition including 3 to 10% by weight of carboxymethyl cellulose, 0.1 to 1% by weight of ammonium chloride (NH₄Cl), 0.1 to 1% by weight of magnesium chloride (MgCl₂), 0.1 to 1% by weight of calcium chloride (CaCl₂), 0.1 to 1% by weight of monopotassium phosphate (KH₂PO₄), 0.2 to 1.5% by weight of yeast extract, and the balance of distilled water with 1 to 5 parts by weight of dry agar to obtain a liquid mixture, sterilizing the liquid mixture under pressure, plating the sterilized liquid mixture on a plastic Petri dish, and coagulating the plated liquid mixture.

Next, in step (C), the highly furfural-resistant strain is cultured in the solid medium spread with the diluted soil supernatant at 25 to 35° C. for 2 to 5 days and is then isolated.

The highly furfural-resistant strain is Enterobacter cloacae strain GGT036 [Accession No. KCTC 12672BP].

For example, furfural as a toxic substance has a half maximal inhibitory concentration (IC₅₀) value of 45 to 55 mM, preferably 47 to 50 mM, against the Enterobacter cloacae strain GGT036 [Accession No. KCTC 12672BP]. The term “IC₅₀” refers to the concentration of the toxic substance required to inhibit the maximum growth of the microorganism by 50%. A higher IC₅₀ value indicates that the strain grows better even at a higher concentration of the toxic substance.

Furfural causes low fermentation productivity due to its toxicity. Thus, removal of the toxic substance is required before application to fermentation. The Enterobacter cloacae strain GGT036 [Accession No. KCTC 12672BP] can be directly used in the fermentation process without removal of the toxic substance due to its high furfural resistance.

The present invention also provides a method for producing bioenergy using the Enterobacter cloacae strain GGT036 [Accession No. KCTC 12672BP].

The method of the present invention includes inoculating the Enterobacter cloacae strain GGT036 [Accession No. KCTC 12672BP] alone or its mixture with another strain such as Clostridium tyrobutyricum or Saccharomyces cerevisiae into a fermentation medium supplemented with a lignocellulosic hydrolysate as a carbon source and fermenting the lignocellulosic hydrolysate. The method of the present invention enables the production of metabolites, such as lactic acid, succinic acid, and sodium L-glutamate (MSG), as well as chemicals, such as biofuels for bioenergy production, and plastics.

Specifically, the fermentation medium is prepared by adding 0.1 to 5 parts by weight of yeast extract, 0.01 to 1 part by weight of magnesium sulfate, 0.01 to 1 part by weight of manganese sulfate, 0.01 to 1 part by weight of iron sulfate, and 0.01 to 1 part by weight of sodium chloride to 100 parts by weight of a lignocellulosic hydrolysate, and sterilizing the mixture under high pressure at 120 to 130 ° C. for 5 to 30 minutes.

The method of the present invention is carried out by batch culture. According to the batch culture, the Enterobacter cloacae strain GGT036 [Accession No. KCTC 12672BP] and Clostridium tyrobutyricum are inoculated into the medium in a serum bottle and cultured in a shaking incubator at 150 to 300 rpm and 35 to 40° C. for 40 to 60 hours.

The lignocellulosic hydrolysate is obtained by hydrolysis of wood. The hydrolysis is performed by any suitable process known in the art.

The following examples are provided to assist in further understanding of the invention. However, these examples are intended for illustrative purposes only. It will be evident to those skilled in the art that various modifications and changes can be made without departing from the scope and spirit of the invention and such modifications and changes are encompassed within the scope of the appended claims.

### PREPARATIVE EXAMPLE 1

### Preparation of Solid Media

5 wt % of carboxymethyl cellulose, 0.3 wt % of ammonium chloride (NH₄Cl), 0.3 wt % of magnesium chloride (MgCl₂), 0.3 wt % of calcium chloride (CaCl₂), 0.3 wt % of monopotassium phosphate (KH₂PO₄), 0.5 wt % of yeast extract, and the balance of distilled water were mixed together to prepare a solid medium composition. 100 parts by weight of the solid medium composition was mixed with 1.5 parts by weight of dry agar powder to obtain a liquid mixture. The liquid mixture was sterilized under pressure at 121° C. for 15 min. Aliquots (each 20 ml) of the sterilized liquid mixture were plated on plastic Petri dishes and coagulated to prepare solid media.

### Example 1

### Isolation of Enterobacter cloacae strain GGT036 [Accession No. KCTC 12672BP]

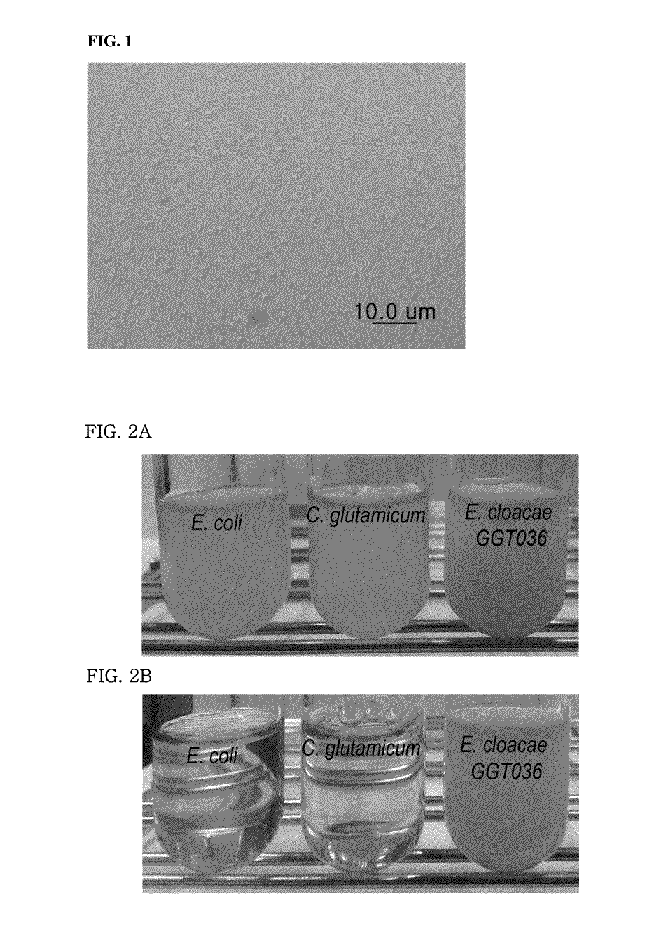

0.1 g of a soil sample taken from Gwanak mountain, Seoul, Korea, was added to 10 mL of a 0.85% NaCl solution as an isotonic solution. After stirring, the soil supernatant was collected and diluted 5-fold with distilled water. The dilution was spread on the solid media prepared in Preparative Example 1, followed by incubation at 30° C. for 3 days. After colonies grown in the solid media were isolated, each colony was streaked on the solid medium. As a result, pure Enterobacter cloacae strain GGT036 was obtained (FIG. 1).

The Enterobacter cloacae strain GGT036 received the accession number of KCTC 12672BP on Aug. 29, 2014.

### TEST EXAMPLES

### Test Example 1

### Identification of Resistance to Furfural

FIG. 2A is an image showing the growth of Enterobacter cloacae strain GGT036, E. coli, and Corynebacterium glutamicum in furfural-free media and FIG. 2B is an image showing the growth of Enterobacter cloacae strain GGT036, E. coli, and Corynebacterium glutamicum in furfural-containing media.

The degrees of growth of Enterobacter cloacae strain GGT036 [Accession No. KCTC 12672BP], E. coli (Comparative Example 1), and Corynebacterium glutamicum (Comparative Example 2) in media containing 30 mM furfural were compared by turbidity measurements to identify the resistance of Enterobacter cloacae strain GGT036 to furfural.

The media used were LB media supplemented with 1% glucose. An observation was made to determine the dependence of the growth of the strains in the media containing 30 mM furfural (FIG. 2B) and the furfural-free media (FIG. 2A) on the presence or absence of furfural.

After 7 mL of each medium was added to a glass tube (24 mm), the colonies obtained in Example 1 were inoculated into the medium, followed by culture at 30° C. for 24 h.

As shown in FIG. 2, all three strains were grown in the furfural-free media (FIG. 2A) but E. coli and Corynebacterium glutamicum, which are currently in use as industrial strains, were not grown in the furfural-containing media and cell growth was observed only for Enterobacter cloacae strain GGT036 (FIG. 2B).

### Test Example 2

### Identification of Degrees of Inhibition of Growth by Furfural

FIG. 3 is a graph showing the degrees of inhibition of growth of Enterobacter cloacae strain GGT036 in the presence of furfural at different concentrations.

LB media were supplemented with 1% glucose as a carbon source to prepare culture media. To the culture media was added furfural at different concentrations of 20 mM, 40 mM, and 60 mM. The growth rates of the strain in the furfural-containing media were compared with the growth rate of the strain in the furfural-free medium.

After 50 mL of each culture medium was added to an Erlenmeyer flask, Enterobacter cloacae strain GGT036 [Accession No. KCTC 12672BP] was inoculated at an initial OD600 of 1, followed by culture at 30 ° C.

As shown in FIG. 3, the growth rate of Enterobacter cloacae strain GGT036 [Accession No. KCTC 12672BP] was slightly retarded with increasing furfural concentration from 0 mM to 40 mM. For the medium containing 60 mM furfural, the OD value did not exceed 2 even after 30 h of culturing, indicating that there was no substantial growth of the strain.

The half maximal inhibitory concentration (IC₅₀) values of furfural were calculated. As a result, the IC₅₀ value against Enterobacter cloacae strain GGT036 [Accession No. KCTC 12672BP] (47.7 mM) was about 2 and about 4 times higher than the IC₅₀ values against E. coli (24.9 mM) and Corynebacterium glutamicum (10 mM), respectively. These results demonstrate higher furfural resistance of Enterobacter cloacae strain GGT036. Taking into consideration that the degree of inhibition of cell growth decreases exponentially with increasing furfural concentration, it was also demonstrated that Enterobacter cloacae strain GGT036 [Accession No. KCTC 12672BP] has high resistance to furfural.

### Test Example 3

### Identification of Degree of Conversion of Furfural to Furfuryl Alcohol Depending on Furfural Concentration

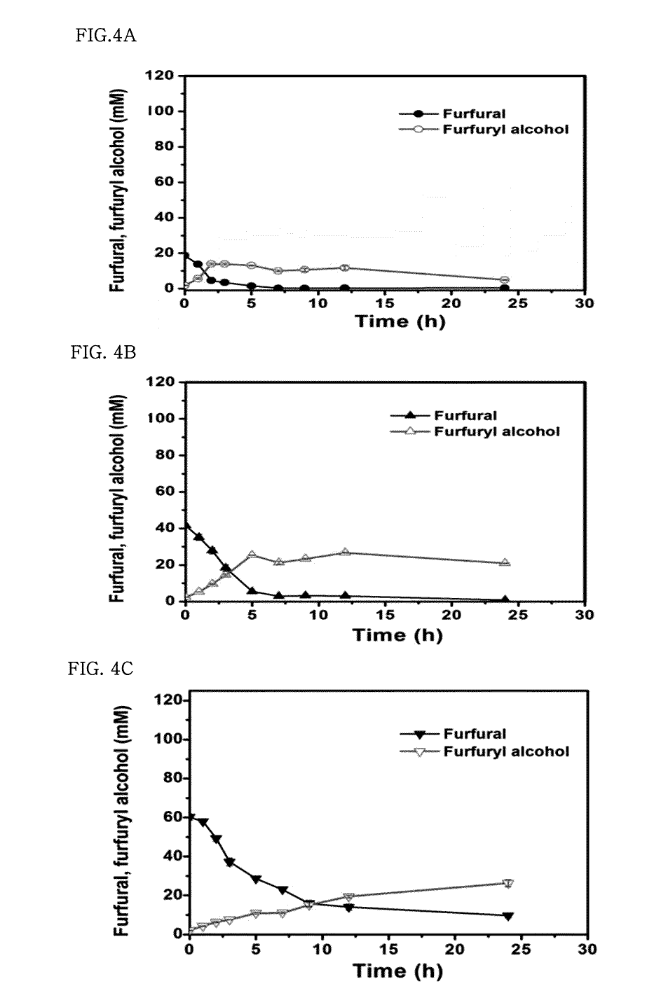

FIG. 4A is a graph showing changes in the concentration of furfural and furfuryl alcohol in a culture medium containing 20 mM furfural when Enterobacter cloacae strain GGT036 was allowed to grow in the culture medium, FIG. 4B is a graph showing changes in the concentration of furfural and furfuryl alcohol in a culture medium containing 40 mM furfural when Enterobacter cloacae strain GGT036 was allowed to grow in the culture medium, and FIG. 4C is a graph showing changes in the concentration of furfural and furfuryl alcohol in a culture medium containing 60 mM furfural when Enterobacter cloacae strain GGT036 was allowed to grow in the culture medium.

The concentrations of furfural and furfuryl alcohol were measured by GC. The culture media were sampled at the times indicated in the graphs. After every sampling, the culture medium was centrifuged at 12000 rpm for 5 min. Only the supernatant was collected, filtered, and used for analysis.

As shown in FIG. 4, as the culture proceeded, the concentration of furfural decreased, resulting in an increase in the concentration of furfuryl alcohol. For both 20 mM (FIG. 4A) and 40 mM (FIG. 4B), complete removal of furfural from the culture media was observed. For 60 mM (FIG. 4C), furfural was present at a concentration as low as about 10 mM.

The strain of the present invention was deposited with the Korean Collection for Type Culture (KCTC) under Accession No. KCTC 12672BP on Aug. 28, 2014.

What is claimed is:

1. A method for isolating highly furfural-resistant Enterobacter cloacae strain GGT036 [Accession No. KCTC 12672BP], comprising
(A) mixing soil with an isotonic solution and collecting the soil supernatant from the mixture,
(B) diluting the soil supernatant and spreading the dilution on a medium supplemented with carboxymethyl cellulose as a nutrient source, and
(C) growing a desired strain by culture in the medium spread with the diluted soil supernatant and isolating the strain.

2. The method according to claim 1, wherein the medium is supplemented with 3 to 10% by weight of carboxymethyl cellulose as a nutrient source.

3. Highly furfural-resistant Enterobacter cloacae strain GGT036 [Accession No. KCTC 12672BP].

4. The highly furfural-resistant Enterobacter cloacae strain GGT036 [Accession No. KCTC 12672BP] according to claim 3, wherein furfural has an IC₅₀ value of 45 to 55 mM against the Enterobacter cloacae strain GGT036 [Accession No. KCTC 12672BP].

5. The highly furfural-resistant Enterobacter cloacae strain GGT036 [Accession No. KCTC 12672BP] according to claim 3, wherein the Enterobacter cloacae strain GGT036 [Accession No. KCTC 12672BP] grows in a medium supplemented with carboxymethyl cellulose as a nutrient source.
